(19) United States
(12) Patent Application Publication
(10) Pub. No.: US 2007/0173528 A1
(43) Pub. Date: Jul. 26, 2007

(54) Process for preparing solifenacin

(76) Inventors: Nurit Perlman, Kfar Saba (IL); Tamar Nidam, Yehud (IL)

Correspondence Address: KENYON & KENYON LLP, ONE BROADWAY, NEW YORK, NY, 10004, US

(21) Appl. No.: 11/645,021

(22) Filed: Dec. 21, 2006

Related U.S. Application Data
(60) Provisional application No. 60/753,236, filed on Dec. 21, 2005.
(60) Provisional application No. 60/835,802, filed on Aug. 3, 2006.
(60) Provisional application No. 60/860,642, filed on Nov. 22, 2006.
(60) Provisional application No. 60/873,022, filed on Dec. 6, 2006.

(51) Int. Cl. A61K 31/4745; A61K 31/47; C07D 453/02; C07D 217/02

(52) U.S. Cl. 514/305; 514/310; 546/146; 546/137

(57) ABSTRACT

Provided are new intermediates of solifenacin and methods for their preparation, as well as methods of preparing solifenacin and solifenacin succinate.

### CROSS-REFERENCE TO RELATED APPLICATIONS

This application claims the benefit of priority to U.S. Provisional Patent Application Nos. 60/753,236, filed Dec. 21 2005; 60/835,802, filed Aug. 3, 2006; 60/860,642, filed Nov. 22, 2006; and 60/873,022, filed Dec. 6, 2006, each of which is hereby incorporated by reference in their entirety.

### FIELD OF THE INVENTION

The present invention relates to new intermediates of solifenacin, methods for their preparation, and novel methods for preparing solifenacin and solifenacin succinate.

### BACKGROUND OF THE INVENTION

(3R)-1 -azabicyclo[2.2.2]oct-3-yl-(1S)-1-phenyl-3,4-dihydroisoquinoline-2-(1H)-carboxylate ((S)-phenyl-1,2,3,4-tetrahydroisoquinoline-2-carboxylic acid 3(R)-quinuclidinyl ester) is known as solifenacin, also known as YM-905 (in its free base form) and YM-67905 (in its succinate form). Solifenacin has the molecular formula C₂₃H₂₆O₂, a molecular weight of 362.4647, and the following chemical structure:

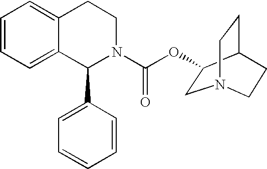

C₂₃H₂₆N₂O₂

Exact Mass: 362.1994

Mol. Wt.: 362.4647

m/e: 362.1994 (100.0%), 363.2028 (25.6%), 364.2061 (3.1%) C, 76.21 ; H, 7.23 ; N, 7.73; O, 8.83.

Solifenacin succinate is a urinary antispasmodic, acting as a selective antagonist to the M(3)-receptor. It is used as treatment of symptoms of overactive bladder, such as urinary urgency and increased urinary frequency, as may occur in patients with overactive bladder syndrome (OAB), as reviewed in Chilman-Blair, Kim et al., Drugs of Today, 40(4):343-353 (2004). Its crystalline powder is white to pale yellowish-white and is freely soluble at room temperature in water, glacial acetic acid, DMSO, and methanol.

The commercial tablet is marketed under the trade name VESICARE®. As VESICARE®, it was approved by the FDA for once daily treatment of OAB and is prescribed as 5 mg and 10 mg tablets.

The drug was developed by Yamanouchi Pharmaceutical Co. Ltd. and disclosed in U.S. Pat. No. 6,017,927 and its continuation, U.S. Pat. No. 6,174,896. Disclosed therein are compounds whose general formula is: 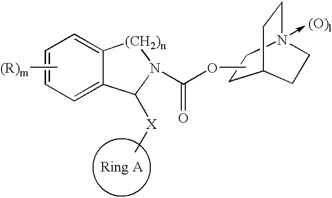
The definitions of the various groups encompass solifenacin, including its salts, as well as pharmaceutical compositions. WO 2005/087231 arid WO 2005/105795 more specifically disclose processes for the production of solifenacin and its salt to a high degree of purity for medicinal use.

There are two principal processes for synthesizing solifenacin disclosed in the art. Both use the following as key starting materials: 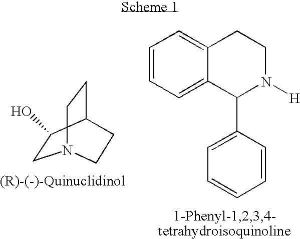
wherein the quinuclidinol reactant is available commercially. The overall synthesis as reported by Mealy, N., et al. in Drugs of the Future, 24 (8): 871-874 (1999) is depicted in Scheme 2:

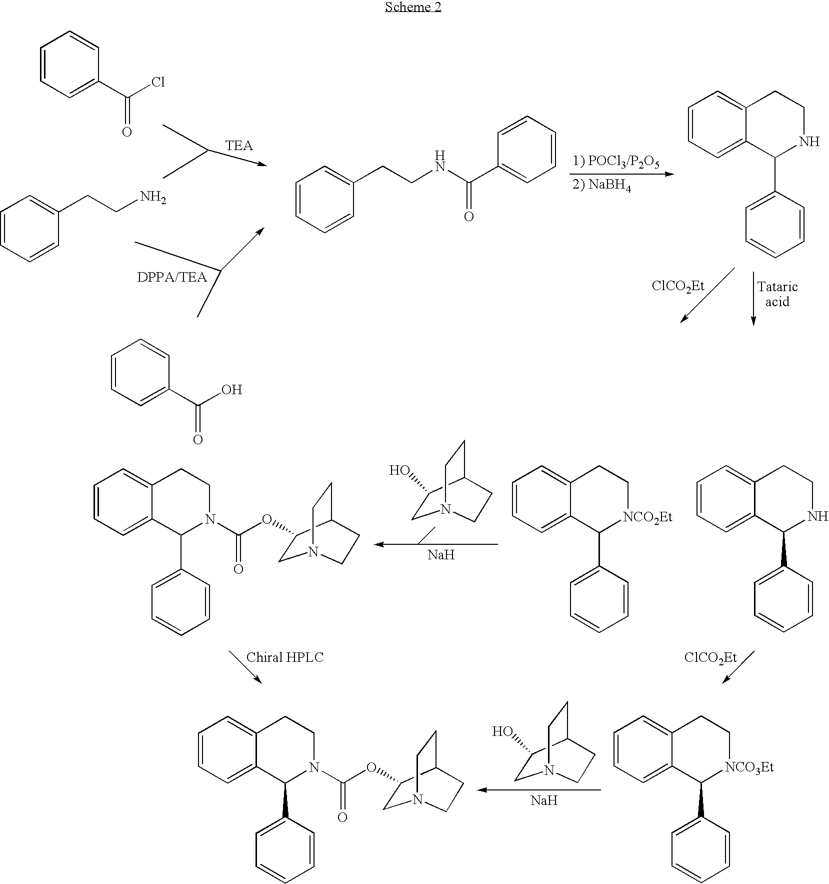

U.S. Pat. No. 6,017,927 discloses another process for the preparation of solifenacin, wherein 3-quinuclidinyl chloroformate monohydrochloride is admixed with (1R)-1-phenyl-1,2,3,4-tetrahydroisoquinoline to obtain solifenacin, as seen below in Scheme 3:

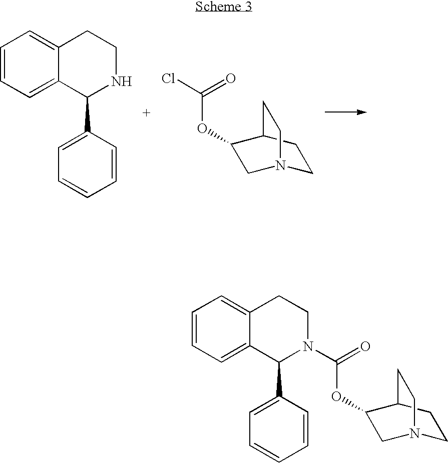

There is a need in the art for additional processes for preparing solifenacin that employ shorter reaction times and less hazardous materials.

### SUMMARY OF THE INVENTION

In one embodiment, the invention encompasses a haloalkyl-1,2,3,4-tetrahydroisoquinoline carbamate of the formula 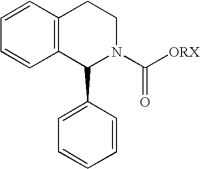
wherein R is an alkyl and X is a halogen.

In another embodiment, the invention encompasses a process for preparing a haloalkyl-1,2,3,4-tetrahydroisoquinoline carbamate of the formula 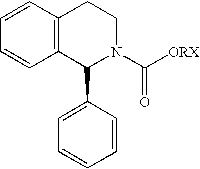
comprising combining (S)-1-phenyl-1,2,3,4-tetrahydroisoquinoline, a haloalkylhalofonnate of the formula 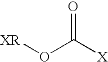
and a base to obtain the haloalkyl-1,2,3,4-tetrahydroisoquinoline carbamate, wherein R is an alkyl and X is a halogen.

In another embodiment, the invention encompasses a process for preparing solifenacin comprising: combining (S)-1-phenyl-1,2,3,4-tetrahydroisoquinoline, a haloalkylhaloformate of the formula 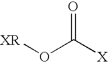
and a base to obtain a haloalkyl-1,2,3,4-tetrahydroisoquinoline carbamate of the formula 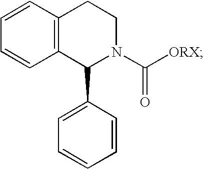
and converting the haloalkyl-1,2,3,4-tetrahydroisoquinoline carbamate into solifenacin, wherein R is an alkyl and X is a halogen.

In another embodiment, the invention encompasses a process for preparing solifenacin comprising: combining (S)-1-phenyl-1,2,3,4-tetrahydroisoquinoline, a haloalkylhaloformate of the formula 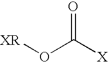
and a first base to obtain a haloalkyl-1,2,3,4-tetrahydroisoquinoline carbamate of the formula 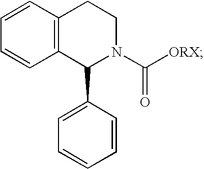
and combining the haloalkyl-1,2,3,4-tetrahydroisoquinoline carbamate with (R)-3-quinuclidinol in the presence of a second base to obtain solifenacin.

In one embodiment, the invention encompasses a haloalkyl-quinuclidyl-carbonate of the formula 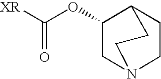
wherein R is an alkyl and X is a halogen.

In another embodiment, the invention encompasses a process for preparing a haloalkyl-quinuclidyl-carbonate of the formula 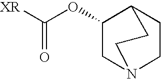
comprising combining (R)-3-quinuclidinol, a haloalkylhaloformate of the formula 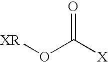
and a base to obtain the haloalkyl-quinuclidyl-carbonate, wherein R is an alkyl and X is a halogen.

In another embodiment, the invention encompasses a process for preparing solifenacin comprising: comprising combining (R)-3-quinuclidinol, a haloalkylhaloformate of the formula 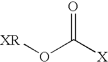
and a base to obtain a haloalkyl-quinuclidyl-carbonate of the formula 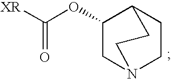
and converting the haloalkyl-quinuclidyl-carbonate into solifenacin, wherein R is an alkyl and X is a halogen.

In another embodiment, the invention encompasses a process for preparing solifenacin comprising: combining (R)-3-quinuclidinol, a haloalkylhaloformate of the formula 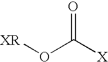
and a first base to obtain a haloalkyl-quinuclidyl-carbonate of the formula 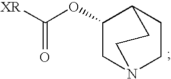
and combining the haloalkyl-quinuclidyl-carbonate with (S)-1-phenyl-1,2,3,4-tetrahydroisoquinoline and a second base to obtain solifenacin, wherein R is an alkyl and X is a halogen.

In another embodiment, the invention encompasses a process for preparing solifenacin succinate comprising preparing solifenacin by one of the above-described processes, and converting the solifenacin into solifenacin succinate.

### DETAILED DESCRIPTION OF THE INVENTION

As used herein, the term “room temperature” refers to a temperature of about 20° C. to about 25° C.

The present invention provides new intermediates of solifenacin, and improved processes for the preparation of solifenacin succinate and solifenacin using (S)-1-phenyl-1,2,3,4-tetrahydroisoquinoline (S-IQL), haloalkylhaloformate and (R)-3-quinuclidinol.

The present invention provides haloalkyl-IQL-carbamate. Preferably, the haloalkyl-IQL-carbamate is chloroethyl-IQL-carbamate.

The present invention provides a process for the preparation of haloalkyl-IQL-carbamate comprising combining (S)-1-phenyl-1,2,3,4-tetrahydroisoquinoline (S-IQL), haloalkylhaloformate and a first base.

Preferably, the process further comprises adding a first organic solvent.

Preferably, the first organic solvent is selected from the group consisting of: dimethylformamide (DMF), tetrahydrofuran (THF), methyl-THF, dioxane, dimethylsulfoxide (DMSO), aromatic hydrocarbon, dichloromethane and mixtures of them with water. More preferably, the first organic solvent is selected from the group consisting of: aromatic hydrocarbon and THF. Preferably, the aromatic hydrocarbon is selected from the group consisting of toluene and xylene. Most preferably, the first organic solvent is toluene.

Preferably, the haloalkylhaloformate is selected from the group consisting of fluoroethylchloroformate, chloroethylbromoformate and bromoethylchloroformate, more preferably, chloroethylchloroformate.

Preferably, the process comprises: combining (S)-1-phenyl-1,2,3,4-tetrahydroisoquinoline (S-IQL), a first organic solvent and a first base, and thereafter combining the haloalkylhaloformate to obtain haloalkyl-IQL-carbamate. Preferably, the haloalkylhaloformate is added to the combination of the (S)-1-phenyl-1,2,3,4-tetrahydroisoquinoline (S-IQL), the first organic solvent and the first base. Preferably, the haloalkylhaloformate is added dropwise. Preferably, prior to the haloalkylhaloformate addition, a cooling step is performed. Preferably, the cooling is to a temperature of about 0° C. to about 25° C.

Preferably, the temperature during the process is from about 0° to about 25° C.

Preferably, the first base is an organic base or carbonate. Preferably, the organic base is an amine. Preferably, the amine is selected from the group consisting of diisopropylamine and triethylamine. Preferably, the carbonate is selected from the group consisting of sodium carbonate, potassium carbonate, sodium bicarbonate, and potassium bicarbonate.

After combining (S)-1-phenyl-1,2,3,4-tetrahydroisoquinoline (S-IQL), haloalkylhaloformate, and a first base, a reaction mixture is obtained. Preferably, the reaction mixture is maintained, preferably for about 1 hour to about 10 hours.

Preferably, the process further comprises separating the haloalkyl-IQL-carbamate. Preferably, the separation is by filtration. Optionally, the separation isolation is by extraction with water and evaporation of the solvent.

The present invention is also directed to the synthesis of solifenacin succinate by converting the haloalkyl-IQL-carbamate obtained by the above process to solifenacin succinate.

The present invention provides a process for the preparation of solifenacin, comprising of the steps:
(a) combining (S)-1-phenyl-1,2,3,4-tetrahydroisoquinoline (S-IQL), haloalkylhaloformate, and a first base to obtain haloalkyl-IQL-carbamate; and
(b) combining the haloalkyl-IQL-carbamate with (R)-3-quinuclidinol in the presence of a second base to obtain solifenacin.

The above process may be illustrated in the following Scheme 4:

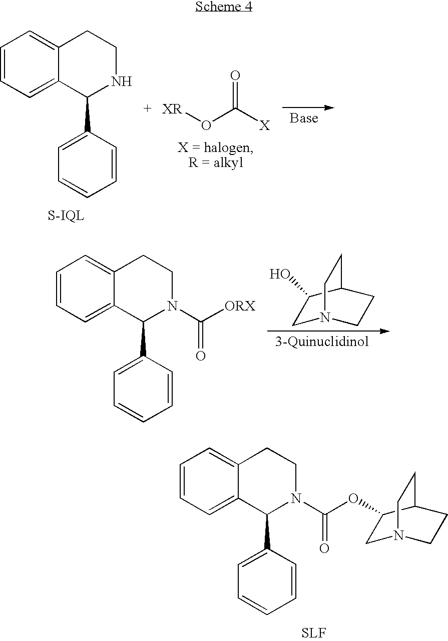

Preferably, step (a) further comprises adding a first organic solvent as described above.

Preferably, the haloalkylhaloformate is as described above.

Preferably, step (a) first comprises combining (S)-1-phenyl-1,2,3,4-tetrahydroisoquinoline (S-IQL), a first organic solvent and a first base, and thereafter combining the haloalkylhaloformate to obtain haloalkyl-IQL-carbamate, as described above.

Preferably, the temperature in step (a) is as described above.

Preferably, the first base in step (a) is as described above.

After combining (S)-1-phenyl-1,2,3,4-tetrahydroisoquinoline (S-IQL), haloalkylhaloformate, and a first base, a reaction mixture is obtained. Preferably, the reaction mixture is maintained, as described above.

Optionally, prior to step (b), the haloalkyl-IQL-carbamate of step (a) is separated. Preferably, the separation is by filtration. Optionally, the separation isolation is by extraction with water and evaporation of the solvent.

Preferably, step (b) further comprises adding a second organic solvent. Preferably, the second organic solvent in step (b) is selected from the group consisting of, dimethylformamide (DMF), tetrahydrofuran (THF), methyl-THF, dioxane, dimethylsulfoxide (DMSO), aromatic hydrocarbon, and mixtures thereof. More preferably, the second organic solvent in step (b) is selected from the group consisting of aromatic hydrocarbon and DMF. Preferably, the aromatic hydrocarbon is selected from the group consisting of toluene and xylene. Most preferably, the second organic solvent in step (b) is toluene.

Preferably, the temperature in step (b) is from about 10° to about 100° C. More preferably, the temperature in step (b) is from about 70° to about 90° C.

Preferably, the second base in step (b) is selected from the group consisting of: metal alkyls, metal alkoxides and sodium hydride. More preferably, the second base in step (b) is sodium hydride.

Optionally, step (b) further comprises distilling the solvent.

After combining the haloalkyl-IQL-carbamate with (R)-3-quinuclidinol in the presence of a second base, a reaction mixture is obtained. Preferably, the reaction mixture is maintained, preferably for about 1 hour to about 24 hours.

Preferably, the process further comprises a recovery step.

Preferably, the recovery comprises: extracting solifenacin with a saturated NaCl solution, removing the aqueous layer, adding HCl solution to a obtain a two phase system, separating the aqueous phase, basifying the aqueous phase with K₂CO₃ solution, extracting it with EtOAc and isolating. Preferably, the isolation is by filtering and evaporating the organic solvent.

The present invention provides haloalkyl-quinuclidyl-carbonate. Preferably, the haloalkyl-quinuclidyl-carbonate is chloroethyl-quinuclidyl-carbonate.

The present invention provides a process for the preparation of haloalkyl-quinuclidyl-carbonate, comprising combining (R)-3-quinuclidinol, haloalkylhaloformate and a first base.

Preferably, the process further comprises adding a first organic solvent. Preferably, the first organic solvent is selected from the group consisting of, dimethylformamide (DMF), tetrahydrofuran (THF), methyl-THF, dioxane, dimethylsulfoxide (DMSO), aromatic hydrocarbon, and dichloromethane. More preferably, the first organic solvent is selected from the group consisting of aromatic hydrocarbon and THF. Preferably, the aromatic hydrocarbon is selected from the group consisting of toluene and xylene. Preferably, the first organic solvent is toluene.

Preferably, the haloalkylhaloformate is selected from the group consisting of haloalkylbromoformate or haloalkylchloroformate, preferably fluoroethylchloroformate and chloroethylchloroformate, more preferably, chloroethylchloroformate.

Preferably, the temperature during the process is from about 0° to about 25° C.

Preferably, the first base is an organic base. Preferably, the organic base is an amine. Preferably, the amine is selected from the group consisting of diisopropylamine and triethylamine.

After combining (R)-3-quinuclidinol, haloalkylhaloformate and a first base, a reaction mixture is obtained. Preferably, the reaction mixture is maintained, preferably for about 1 hour to about 10 hours.

Preferably, the process further comprises separating the haloalkyl-quinuclidyl-carbonate. Preferably, the separation is by filtration.

The present invention is also directed to the synthesis of solifenacin succinate by converting the haloalkyl-quinuclidyl-carbonate obtained by the above process to solifenacin succinate.

The present invention provides another process for the preparation of solifenacin, comprising of the steps:
(a) combining (R)-3-quinuclidinol, haloalkylhaloformate and a first base to obtain haloalkyl-quinuclidyl-carbonate; and
(b) combining the haloalkyl-quinuclidyl-carbonate with (S)-1-phenyl-1,2,3,4-tetrahydroisoquinoline (S-IQL) and a second base to obtain solifenacin.

The above process may be illustrated in the following Scheme 5:

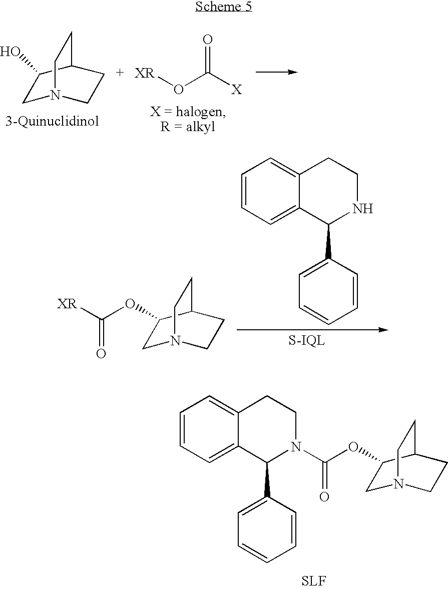

Preferably, step (a) further comprises adding a first organic solvent as described above.

Preferably, the haloalkylhaloformate is as described above.

Preferably, the temperature in step (a) is as described above.

Preferably, the first base in step (a) is as described above.

After combining (R)-3-quinuclidinol, haloalkylhaloformate and a first base, a reaction mixture is obtained. Preferably, the reaction mixture is maintained, as described above.

Optionally, prior to step (b), the haloalkyl-quinuclidyl-carbonate of step (a) is separated. Preferably, the separation is by filtration.

Preferably, step (b) further comprises adding a second organic solvent.

Preferably, the second organic solvent in step (b) is selected from the group consisting of: dimethylformamide (DMF), tetrahydrofuran (THF), methyl-THF, dioxane, dimethylsulfoxide (DMSO), aromatic hydrocarbon, and dichloromethane. More preferably, the second organic solvent in step (b) is selected from the group consisting of aromatic hydrocarbon and THF. Preferably, the aromatic hydrocarbon is selected from the group consisting of toluene and xylene.

Preferably, the temperature in step (b) is from about 10° to about 100° C. More preferably, the temperature in step (b) is from about 70° to about 90° C.

Preferably, the second base is selected from the group consisting of: metalalkyls, metal alkoxides and sodium hydride. More preferably, the second organic base is sodium hydride.

After combining the haloalkyl-quinuclidyl-carbonate with (S)-1-phenyl-1,2,3,4-tetrahydroisoquinoline (S-IQL) and a second base, a reaction mixture is obtained. Preferably, the reaction mixture is maintained, preferably for about 1 hour to about 24 hours.

Preferably, the process further comprises a recovery step. Preferably, the recovery comprises: extracting solifenacin with a saturated NaCl solution, removing the aqueous layer, adding HCI solution to a obtain a two phase system, separating the aqueous phase, basifying the aqueous phase with K₂CO₃ solution, extracting it with EtOAc and isolating. Preferably, the isolation is by filtering and evaporating the organic solvent.

The present invention is also directed to the synthesis of solifenacin succinate by converting the solifenacin obtained by the above processes to solifenacin succinate. The conversion of the solifenacin to solifenacin succinate may be performed by any method known to one of skill in the art. Such methods include, but are not limited to, that disclosed in WO 2005/087231, hereby incorporated by reference.

Preferably, the conversion of the solifenacin to solifenacin succinate is performed by dissolving the solifenacin in EtOH and adding succinic acid to obtain a precipitate of solifenacin succinate. Optionally, the solution may be seeded with solifenacin succinate to induce the precipitation of the solifenacin succinate.

Having described the invention with reference to certain preferred embodiments, other embodiments will become apparent to one skilled in the art from consideration of the specification. The invention is further defined by reference to the following examples describing in detail the preparation of the composition and methods of use of the invention. It will be apparent to those skilled in the art that many modifications, both to materials and methods, may be practiced without departing from the scope of the invention.

### EXAMPLES

### Example 1

Preparation of Solifenacin Succinate

A solution of (S)-1-phenyl-1,2,3,4-tetrahydroisoquinoline (C₁₅H₁₅N) (16 g), toluene (80 ml), and diisopropylethylamine (DIPEA, 13.5 g) was cooled to 0° C. Chloroethylchloroformate (C₃H₄Cl₂O₂) (CECF, 13.Ogr) was added dropwise, keeping the temperature between 0°-20° C. After stirring at room temperature for 1.5 hours, the mixture was filtered.

The filtrate was added to solution of (R)-quinuclidin-3-ol (C₇H₁₃NO) (11.6 g) in toluene (80 ml), DMF (16 ml), and NaH (60%, 5.5 g) at 80° C. during 1 hour, and stirred at 95°-100° C. for 17 hours. The mixture was cooled to room temperature, and THF (small amount) was added. A saturated NaCl solution (300 ml) was added, and the phases were separated. The organic phase was acidified with 10% HCl solution, and the phases were separated. The aqueous phase was basified with K₂CO₃ solution and extracted with ethyl acetate (EtOAc). The organic phase was filtered and evaporated to obtain solifenacin (SLF) (21.25 g). The residue was dissolved in ethanol (EtOH) (100 ml) and succinic acid (7.0 g) was added. Seeding with SLF-succinate was performed, and the mixture was stirred at RT for 16 hours. The product was isolated by vacuum filtration, washed with EtOH (3×20 ml), and dried in vacuum oven at 500 over night to obtain SLF-succinate (10.46 g).

### Example 2

Preparation of Solifenacin Succinate

Chloroethylchloroformate (CECF, 13.0 g) is added dropwise to solution of (R)-quinuclidin-3-ol (11.6 g) and diisopropylethylamine (DIPEA, 13.5 g) in THF (150 ml), keeping the temperature between 0°-20° C. The mixture is stirred at room temperature for several hours. Then (S)-1-phenyl-1,2,3,4-tetrahydroisoquinoline (16 g) is added and the solution is stirred at room temperature for another 16 hours. The solution is diluted with EtOAc (350 ml) and washed with a saturated NaCl solution (300 ml). The organic phase is acidified with 10% HCl solution, and the phases are separated. The aqueous phase is basified with K₂CO₃ solution and extracted with EtOAc. The organic phase is filtered and evaporated to obtain SLF. The residue is dissolved in EtOH (100 ml), and succinic acid (7.0 g) is added. Seeding with SLF-succinate is performed, and the mixture is stirred at RT for 16 hours. The product is isolated by vacuum filtration, washed with EtOH (3×20 ml), and dried in vacuum oven at 50° over night to obtain SLF-succinate.

### Example 3

Preparation of Solifenacin Succinate

Chloroethylchloroformate (CECF, 13.0 g) is added dropwise to solution of (R)-quinuclidin-3-ol (11.6 g) and diisopropylethylamine (DIPEA, 13.5 g) in Toluene (150 ml), keeping the temperature between 0°-20° C. The mixture is stirred at room temperature for several hours and filtrated. Then (S)-1-phenyl-1,2,3,4-tetrahydroisoquinoline (16 g) is added followed by addition of sodium hydride (60%, 5.5 g) and the mixture is stirred at reflux for another 16hours. The solution is diluted with EtOAc (350 ml) and washed with a saturated NaCl solution (300 ml). The organic phase is acidified with 10% HCl solution, and the phases are separated. The aqueous phase is basified with K2C03 solution and extracted with EtOAc. The organic phase is filtered and evaporated to obtain SLF. The residue is dissolved in EtOH (100 ml), and succinic acid (7.0 g) is added. Seeding with SLF-succinate is performed, and the mixture is stirred at RT for 16 hours. The product is isolated by vacuum filtration, washed with EtOH (3×20 ml), and dried in vacuum oven at 50° over night to obtain SLF-succinate.

1. A haloalkyl-1,2,3,4-tetrahydroisoquinoline carbamate of the formula 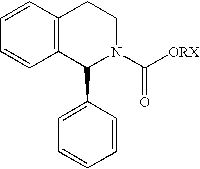
wherein R is an alkyl and X is a halogen.

2. The haloalkyl-1,2,3,4-tetrahydroisoquinoline carbamate of claim 1, wherein R is ethyl.

3. The haloalkyl-1,2,3,4-tetrahydroisoquinoline carbamate of claim 2, wherein X is chlorine.

4. A process for preparing the haloalkyl-1,2,3,4-tetrahydroisoquinoline carbamate of claim 1 comprising combining (S)-1-phenyl-1,2,3,4-tetrahydroisoquinoline, a haloalkylhaloformate of the formula 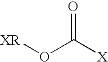
and a base to obtain the haloalkyl-1,2,3,4-tetrahydroisoquinoline carbamate, wherein R is an alkyl and X is a halogen.

5. A process for preparing solifenacin comprising:
a) combining (S)-1-phenyl-1,2,3,4-tetrahydroisoquinoline, a haloalkylhaloformate of the formula 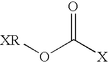
and a base to obtain a haloalkyl-1,2,3,4-tetrahydroisoquinoline carbamate of the formula 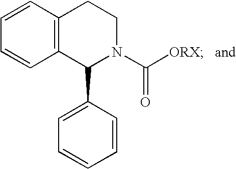
b) converting the haloalkyl-1,2,3,4-tetrahydroisoquinoline carbamate into solifenacin,
wherein R is an alkyl and X is a halogen.

6. A process for preparing solifenacin comprising:
a) combining (S)-1 -phenyl-1,2,3,4-tetrahydroisoquinoline, a haloalkylhaloformate of the formula 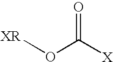
and a first base to obtain a haloalkyl-1,2,3,4-tetrahydroisoquinoline carbamate of the formula 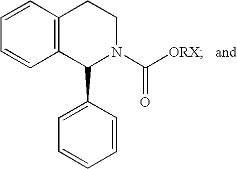
b) combining the haloalkyl-1,2,3,4-tetrahydroisoquinoline carbamate with (R)-3-quinuclidinol in the presence of a second base to obtain solifenacin,
wherein R is an alkyl and X is a halogen.

7. The process of claim 6, further comprising admixing the combination of step a) with a first organic solvent.

8. The process of claim 7, wherein the first organic solvent is selected from the group consisting of dimethylformamide, tetrahydrofuran, methyl-tetrahydrofuran, dioxane, dimethylsulfoxide, an aromatic hydrocarbon, dichloromethane, and mixtures thereof with water.

9. The process of claim 7, wherein the first organic solvent is an aromatic hydrocarbon or tetrahydrofuran.

10-23. (canceled)

24. The process of claim 6, further comprising admixing the combination of step b) with a second organic solvent.

25-33. (canceled)

34. A haloalkyl-quinuclidyl-carbonate of the formula 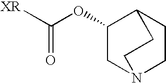
wherein R is an alkyl and X is a halogen.

35. The haloalkyl-quinuclidyl-carbonate of claim 34, wherein R is ethyl.

36. The haloalkyl-quinuclidyl-carbonate of claim 35, wherein X is chlorine.

37. A process for preparing the haloalkyl-quinuclidyl-carbonate of claim 34 comprising combining (R)-3-quinuclidinol, a haloalkylhaloformate of the formula 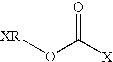
and a base to obtain the haloalkyl-quinuclidyl-carbonate,
wherein R is an alkyl and X is a halogen.

38. A process for preparing solifenacin comprising:
a) comprising combining (R)-3-quinuclidinol, a haloalkylhaloformate of the formula 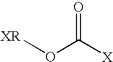
and a base to obtain a haloalkyl-quinuclidyl-carbonate of the formula 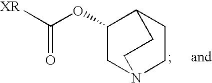
b) converting the haloalkyl-quinuclidyl-carbonate into solifenacin,
wherein R is an alkyl and X is a halogen.

39. A process for preparing solifenacin comprising:
a) combining (R)-3-quinuclidinol, a haloalkylhaloformate of the formula 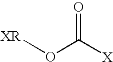
and a first base to obtain a haloalkyl-quinuclidyl-carbonate of the formula 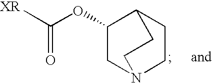
b) combining the haloalkyl-quinuclidyl-carbonate with (S)-1-phenyl-1,2,3,4-tetrahydroisoquinoline and a second base to obtain solifenacin,
wherein R is an alkyl and X is a halogen.

40. The process of claim 39, further comprising admixing the combination of step a) with a first organic solvent.

41-52. (canceled)

53. The process of claim 39, further comprising admixing the combination of step b) with a second organic solvent.

54-61. (canceled)

62. A process for preparing solifenacin succinate comprising:
a) preparing solifenacin by the process of claim 6; and
b) converting the solifenacin into solifenacin succinate.

63. A process for preparing solifenacin succinate comprising:
a) preparing solifenacin by the process of claim 39; and
b) converting the solifenacin into solifenacin succinate.
